(19) United States
(12) Patent Application Publication
(10) Pub. No.: US 2025/0014392 A1
(43) Pub. Date: Jan. 9, 2025

(54) INFORMATION PROCESSING APPARATUS, INFORMATION PROCESSING METHOD, AND NON-TRANSITORY RECORDING MEDIUM

(71) Applicant: NEC Corporation, Minato- ku, Tokyo (JP)

(72) Inventors: Shigeru NAKAMURA, Tokyo (JP); John Kenji David CLARK, Tokyo (JP)

(73) Assignee: NEC Corporation, Minato- ku, Tokyo (JP)

(21) Appl. No.: 18/712,289

(22) PCT Filed: Mar. 31, 2022
(86) PCT No.: PCT/JP2022/016639

§ 371 Date: May 22, 2024

(51) Int. Cl. G06V 40/60; G01S 17/08; G06V 40/13

(52) U.S. Cl. CPC G06V 40/60; G01S 17/08; G06V 40/1318

(57) ABSTRACT

An information processing apparatus includes: a detection unit that detects a target-to-target distance between an imaging target and a light irradiation unit that irradiates light for scanning the imaging target; and a scan control unit that starts irradiation with the light by the light irradiation unit in a case where the target-to-target distance is a desired distance.

### TECHNICAL FIELD

This disclosure relates to technical fields of an information processing apparatus, an information processing method, and a recording medium.

### BACKGROUND ART

Patent Literature 1 describes a non-contact type guide that stops a body properly in a position determined with respect to a sensor, without having a user come into bodily contact with the guide for the sensor, by projecting a pattern indicating the position to the sensor by visible light ray for a portion of the body including biometric information. Patent Literature 2 describes a technique/technology of: dividing a beam of light outputted from a light source into at least one and the other; an optical coherence tomography (OCT) head emitting the divided one light from a light input/output part toward a measurement object and taking in a beam of light reflected by the measurement object as a measuring beam through the light input/output part; rotatably supporting the light input/output part of the OCT head with the measurement as the center; and acquiring an accurate tomography image. Patent Literature 3 describes a technique/technology of: following a change in a tube inner diameter of a tubular body when the tube inner diameter of the tubular body has changed; adjusting a distance between an imaging unit and a laser beam radiation unit in each case; and reliably taking an image of annular laser light. Patent Literature 4 describes a technique/technology of: dividing a tomography image of a fundus captured by an OCT unit into small compartments; obtaining a representative value for each divided small compartments; computing a representative value selected from among the obtained representative values as a quality parameter indicating the quality of a tomogram; and displaying the computed quality parameter of the tomogram in a display. Patent Literature 5 describes an information processing apparatus including: a branching and merging device that branches a light beam emitted from a wavelength sweeping laser light source into an object light beam and a reference light beam; a balance-type light receiver that generates information about a change in an intensity ratio of interference light beams between the object light beam and the reference light beam, the object light beam being scattered from a measurement object after being transmitted through a transparent substrate and then being applied to the measurement object, a structure configured to change a thickness of the transparent substrate being formed on a surface of the transparent substrate; and a control unit that acquires structural data of the measurement object in a depth direction based on information about a change in an intensity ratio of interference light beams between the object light beam scattered from the measurement object and the reference light beam, wherein the control unit connects the acquired plurality of pieces of the structural data in the depth direction with a position of the structure of the transparent substrate as a reference, while moving an irradiation position of the object light.

### CITATION LIST

### Patent Literature

Patent Literature 1: JP2009-251837A
Patent Literature 2: JP2014-178164A
Patent Literature 3: JP2019-060722A
Patent Literature 4: International Publication No. WO2015/098912A1
Patent Literature 5: International Publication No. WO2020/100626A1

### SUMMARY

### Technical Problem

It is an example object of this disclosure to provide an information processing apparatus, an information processing method, and a recording medium that aim to improve the techniques/technologies disclosed in Citation List.

### Solution to Problem

An information processing apparatus according to an example aspect of this disclosure includes: a detection unit that detects a target-to-target distance between an imaging target and a light irradiation unit that irradiates light for scanning the imaging target; and a scan control unit that starts irradiation with the light by the light irradiation unit in a case where the target-to-target distance is a desired distance.

An information processing method according to an example aspect of this disclosure includes: detecting a target-to-target distance between an imaging target and a light irradiation unit that irradiates light for scanning the imaging target; and starting irradiation with the light by the light irradiation unit in a case where the target-to-target distance is a desired distance.

A recording medium according to an example aspect of this disclosure is a recording medium on which a computer program that allows a computer to execute an information processing method is recorded, the information processing method including: detecting a target-to-target distance between an imaging target and a light irradiation unit that irradiates light for scanning the imaging target; and starting irradiation with the light by the light irradiation unit in a case where the target-to-target distance is a desired distance.

### BRIEF DESCRIPTION OF DRAWINGS

FIG. 1 is a block diagram illustrating a configuration of an information processing apparatus in a first example embodiment.

FIG. 2 is a block diagram illustrating a configuration of an information processing apparatus in a second example embodiment.

FIG. 3 is a diagram illustrating a configuration of an optical coherence tomography apparatus in the second example embodiment.

FIG. 4 is a flowchart illustrating a flow of an information processing operation performed by the information processing apparatus in the second example embodiment.

FIG. 5 is a block diagram illustrating a configuration of an information processing apparatus in a third example embodiment.

FIG. 6 is a flowchart illustrating a flow of the information processing operation performed by the information processing apparatus in the third example embodiment.

FIG. 7 is a conceptual diagram illustrating the information processing operation performed by the information processing apparatus in the third example embodiment.

FIG. 8 is a conceptual diagram illustrating the information processing operation performed by an information processing apparatus in a fourth example embodiment.

FIG. 9 is a conceptual diagram illustrating the information processing operation performed by the information processing apparatus in the fourth example embodiment.

FIG. 10 is a block diagram illustrating a configuration of an information processing apparatus in a fifth example embodiment.

FIG. 11 is a flowchart illustrating a flow of the information processing operation performed by the information processing apparatus in the fifth example embodiment.

FIG. 12 is a conceptual diagram illustrating the information processing operation performed by the information processing apparatus in the fifth example embodiment.

### DESCRIPTION OF EXAMPLE EMBODIMENTS

Hereinafter, an information processing apparatus, an information processing method, and a recording medium according to example embodiments will be described with reference to the drawings.

### 1: First Example Embodiment

An information processing apparatus, an information processing method, and a recording medium according to a first example embodiment will be described. The following describes the information processing apparatus, the information processing method, and the recording medium according to the first example embodiment, by using an information processing apparatus 1 to which the information processing apparatus, the information processing method, and the recording medium according to the first example embodiment are applied.

### 1-1: Configuration of Information Processing Apparatus 1

With reference to FIG. 1, a configuration of an information processing apparatus 1 in the first example embodiment will be described. FIG. 1 is a block diagram illustrating the configuration of the information processing apparatus 1 in the first example embodiment.

As illustrated in FIG. 1, the information processing apparatus 1 includes a detection unit 11 and a control unit 12. The detection unit 11 detects a target-to-target distance between an imaging target and a light irradiation unit that irradiates light for scanning the imaging target. The control unit 12 starts irradiation with the light by the light irradiation unit in a case where the target-to-target distance is a desired distance.

### 1-2: Technical Effect of Information Processing Apparatus 1

In the information processing apparatus 1 in the first example embodiment, in a case where the target-to-target distance is a desired distance, i.e., in a case where the imaging target and the light irradiation unit are in an appropriate positional relation, the light irradiation unit starts the irradiation with the light, and it is thus possible to perform high-accuracy optical coherence tomography.

### 2: Second Example Embodiment

An information processing apparatus, an information processing method, and a recording medium according to a second example embodiment will be described. The following describes the information processing apparatus, the information processing method, and the recording medium according to the second example embodiment, by using an information processing apparatus 2 to which the information processing apparatus, the information processing method, and the recording medium according to the second example embodiment are applied.

### 2-1: Configuration of Information Processing Apparatus 2

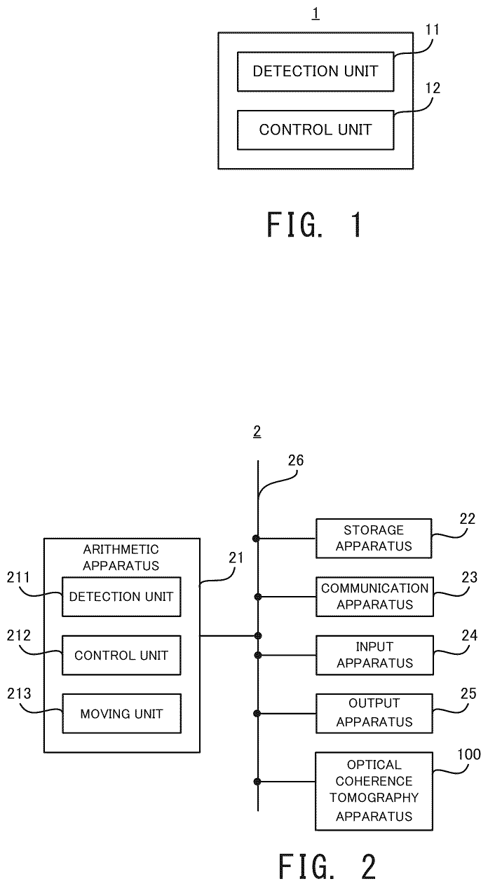

With reference to FIG. 2, a configuration of the information processing apparatus 2 in the second example embodiment will be described. FIG. 2 is a block diagram illustrating the configuration of the information processing apparatus 2 in the second example embodiment.

As illustrated in FIG. 2, the information processing apparatus 2 includes an arithmetic apparatus 21 and a storage apparatus 22. Furthermore, the information processing apparatus 2 may include an optical coherence tomography apparatus 100, a communication apparatus 23, an input apparatus 24, and an output apparatus 25. The information processing apparatus 2, however, may not include at least one of the optical coherence tomography apparatus 100, the communication apparatus 23, the input apparatus 24, and the output apparatus 25. In a case where the information processing apparatus 2 does not include the optical coherence tomography apparatus 100, the information processing apparatus 2 may perform transmission and reception of information through the communication apparatus 23, with the optical coherence tomography apparatus 100. The arithmetic apparatus 21, the storage apparatus 22, the optical coherence tomography apparatus 100, the communication apparatus 23, the input apparatus 24, and the output apparatus 25 may be connected through a data bus 26.

The arithmetic apparatus 21 includes at least one of a CPU (Central Processing Unit), a GPU (Graphics Processing Unit), and a FPGA (Field Programmable Gate Array), for example. The arithmetic apparatus 21 reads a computer program. For example, the arithmetic apparatus 21 may read a computer program stored in the storage apparatus 22. For example, the arithmetic apparatus 21 may read a computer program stored by a computer-readable and non-transitory recording medium, by using a not-illustrated recording medium reading apparatus provided in the information processing apparatus 2 (e.g., the input apparatus 24 described later). The arithmetic apparatus 21 may acquire (i.e., download or read) a computer program from a not-illustrated apparatus disposed outside the information processing apparatus 2, through the communication apparatus 23 (or another communication apparatus). The arithmetic apparatus 21 executes the read computer program. Consequently, a logical functional block for performing an operation to be performed by the information processing apparatus 2 is realized or implemented in the arithmetic apparatus 21. That is, the arithmetic apparatus 21 is allowed to function as a controller for realizing or implementing the logical functional block for performing an operation (in other words, a processing) to be performed by the information processing apparatus 2.

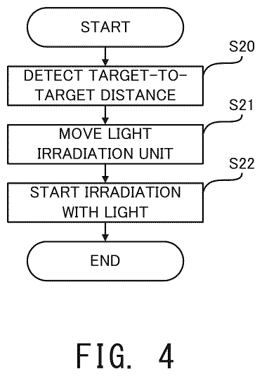

FIG. 2 illustrates an example of the logical functional block realized or implemented in the arithmetic apparatus 21 to perform an information processing operation. As illustrated in FIG. 2, a detection unit 211 that is a specific example of the “detection unit”, a control unit 212 that is a specific example of the “control unit”, and a moving unit 213 that is a specific example of the “moving unit” are realized or implemented in the arithmetic apparatus 21. The arithmetic apparatus 21, however, may not include the moving unit 213. Each operation of the detection unit 211, the control unit 212, and the moving unit 213 will be described later with reference to FIG. 4.

The storage apparatus 22 is configured to store desired data. For example, the storage apparatus 22 may temporarily store a computer program to be executed by the arithmetic apparatus 21. The storage apparatus 22 may temporarily store data that are temporarily used by the arithmetic apparatus 21 when the arithmetic apparatus 21 executes the computer program. The storage apparatus 22 may store data that are stored by the information processing apparatus 2 for a long time. The storage apparatus 22 may include a at least one of a RAM (Random Access Memory), a ROM (Read Only Memory), a hard disk apparatus, a magneto-optical disk apparatus, a SSD (Solid State Drive), and a disk array apparatus. That is, the storage apparatus 22 may include a non-transitory recording medium.

The communication apparatus 23 is configured to communicate with an apparatus external to the information processing apparatus 2 through a not-illustrated communication network. The communication apparatus 23 may be a communication interface based on a standard such as Ethernet (registered trademark), Wi-Fi (registered trademark), and Bluetooth (registered trademark).

The input apparatus 24 is an apparatus that receives an input of information to the information processing apparatus 2 from an outside of the information processing apparatus 2. For example, the input apparatus 24 may include an operating apparatus (e.g., at least one of a keyboard, a mouse trackball, a touch panel, a pointing device such as a pen tablet, a button, and the like) that is operable by an operator of the information processing apparatus 2. For example, the input apparatus 24 may include a reading apparatus that is configured to read information recorded as data on a recording medium that is externally attachable to the information processing apparatus 2.

The output apparatus 25 is an apparatus that outputs information to the outside of the information processing apparatus 2. For example, the output apparatus 25 may output information as an image. That is, the output apparatus 25 may include a display apparatus (a so-called display) that is configured to display an image indicating the information that is desirably outputted. Examples of the display apparatus include a liquid crystal display, an OLED (Organic Light Emitting Diode) display, and the like. For example, the output apparatus 25 may output information as audio/sound. That is, the output apparatus 25 may include an audio apparatus (a so-called speaker) that is configured to output audio/sound. For example, the output apparatus 25 may output information onto a paper surface. That is, the output apparatus 25 may include a print apparatus (a so-called printer) that is configured to print desired information on the paper surface. The input apparatus 24 and the output apparatus 25 may be integrally formed as a touch panel.

The hardware configuration illustrated in FIG. 2 is an example. An apparatus other than the apparatuses illustrated in FIG. 2 may be added, and a part of the apparatuses may not be provided. In addition, a part of the apparatuses may be replaced by another apparatus having a same/similar function. In addition, a part of the functions in the second example embodiment may be provided by another apparatus through a network. The functions in the second example embodiment may be distributed to and realized in a plurality of apparatuses. As described above, the hardware configuration illustrated in FIG. 2 may be changeable as appropriate.

### 2-2: Optical Coherence Tomography Apparatus 100

The optical coherence tomography apparatus 100 irradiates the target with a light beam while performing two-dimensional scanning on the target, performs optical coherence tomography, and generates three-dimensional luminance data on the target.

The optical coherence tomography is a technique/technology of identifying a position in an optical axis direction of a light scattering point where object light is scattered in the target, i.e., in a depth direction of the target, by using interference between the object light and reference light, and acquiring structural data spatially resolved in the depth direction of an inside of the target. The optical coherence tomography technique/technology includes Time Domain (TD-OCT) and Fourier Domain (FD-OCT), but the FD-OCT is employed in the second example embodiment. In the FD-OCT, an interference light spectrum in a wide wavelength band is measured in the interference of the object light and the reference light, and is Fourier-transformed to acquire the structural data in the depth direction. A method of acquiring the interference light spectrum includes Spectral Domain (SD-OCT) using a spectrometer and Swept Source (SS-OCT) using a light source for sweeping a wavelength. The optical coherence tomography apparatus 100 employed in the second example embodiment performs the optical coherence tomography in the SS-OCT.

The optical coherence tomography apparatus 100 may scan an irradiation position of the object light in an in-plane direction perpendicular to the depth direction of an imaging target O (also referred to as a Z direction), thereby to acquire tomography structural data spatially resolved in the in-plane direction and structural data spatially resolved in the depth direction, i.e., three-dimensional tomography structural data on the imaging target O.

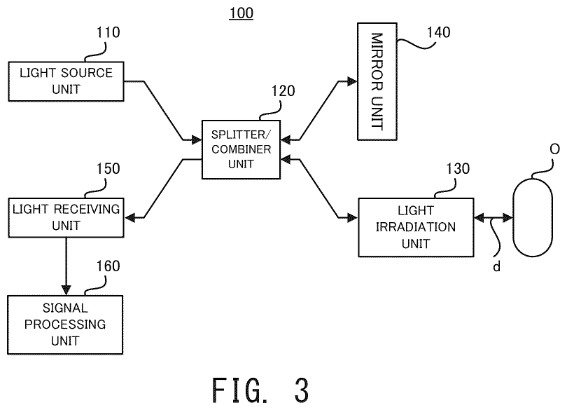

FIG. 3 is a diagram illustrating a schematic configuration of the optical coherence tomography apparatus 100 employed in the second example embodiment. The optical coherence tomography apparatus 100 may image the imaging target O such as a finger of a measurement target person, on the basis of a three-dimensional measurement technique of the optical coherence tomography, thereby to generate three-dimensional luminance data including an inside of a skin. The configuration diagram illustrated in FIG. 3 is merely an example of the apparatus using the optical coherence tomography technique, and the apparatus may have a configuration other than the configuration illustrated in FIG. 3.

As illustrated in FIG. 3, the optical coherence tomography apparatus 100 may include a light source unit 110, a splitter/combiner unit 120, a light irradiation unit 130, a mirror unit 140, a light receiving unit 150, and a signal processing unit 160. The light irradiation unit 130 may include a scanning mirror and a lens. An optical coherence tomography operation of the optical coherence tomography apparatus 100 may be controlled by the arithmetic apparatus 21.

The light source unit 110 may be a laser that emits light while sweeping a wavelength. The light source unit 110 may generate and output a wavelength-swept light pulse. The light source unit 110 may generate the light pulse by sweeping the wavelength from 1250 nm to 1350 nm in a duration of 5 μs, for example.

The splitter/combiner unit 120 may split the light emitted from the light source unit 110 into object light and reference light. The object light may be applied to the imaging target O through the light irradiation unit 130. The object light scattered in the imaging target O may return to the splitter/combiner unit 120. On the other hand, the reference light may be applied to and reflected by the mirror unit 140. The reference light reflected by the mirror unit 140 may return to the splitter/combiner unit 120. The object light scattered by the imaging target O and the reference light reflected by the mirror unit 140 may interfere in the splitter/combiner unit 120, thereby to generate two interference lights. That is, an intensity ratio of the two interference lights may be determined by a phase difference between the object light and the reference light.

The two interference lights may be inputted to the light receiving unit 150, and the light receiving unit 150 may output a voltage corresponding to an intensity difference between the two interference lights. The voltage outputted by the light receiving unit 150 may be inputted to the signal processing unit 160.

### [A Scan]

The signal processing unit 160 may generate interference light spectrum data, on the basis of information about a change in the wavelength of the light emitted by the light source unit 110, and information about a change in the intensity ratio of the two interference lights. The signal processing unit 160 may perform Fourier transform on the generated interference light spectrum data, thereby to acquire data indicating the intensity of backscattered light (object light) at different depth positions in the depth direction (Z direction).

Hereinafter, an operation of acquiring the data indicating the intensity of the backscattered light (object light) in the depth direction (Z direction) of the irradiation position of the object light in the imaging target O, is referred to as “A-scan”. The signal processing unit 160 may be supplied with an A-scan trigger signal from the light source unit 110, and may generate an A-scan waveform for each predetermined period. The signal processing unit 160 may generate a waveform indicating object light backscatter intensity at an Nz point, as the A-scan waveform.

### [B Scan]

The signal processing unit 160 may control the light irradiation unit 130, in accordance with the A-scan trigger signal supplied from the light source unit 110. The light irradiation unit 130 may scan the irradiation position of the object light on the imaging target O. The light irradiation unit 130 may move the irradiation position of the object light in a scanning line direction (also referred to as a “fast axis direction of the scanning” and an “X direction”).

The signal processing unit 160 repeats the A-scan operation for each irradiation position of the object light, and may connect the A-scan waveforms at the respective irradiation positions of the object light. As a result, the signal processing unit 160 may acquire a map of the intensity of two-dimensional backscattered light (object light) in the scanning line direction (X direction) and in the depth direction (Z direction), as a tomography image. Hereinafter, an operation of repeating the A-scan operation while moving in the scanning line direction (the fast axis direction of the scanning, the X direction) and connecting measurement results, is referred to as “B-scan”. In a case where the irradiation position of the object light for each B scan is an Nx point, the tomography image by the B scan is two-dimensional luminance data indicating the object light backscatter intensity at Nz×Nx points.

### [C Scan]

The light irradiation unit 130 may move the irradiation position of the object light not only in the scanning line direction (X direction), but also in a direction perpendicular to the scanning line (also referred to as a “slow axis direction of the scanning” and a “Y direction”). The signal processing unit 160 may repeat the B-scan operation and may connect B-scan measurement results. In this way, the signal processing unit 160 may acquire three-dimensional tomography structural data. Hereinafter, an operation of repeating the B scan operation while moving in the direction perpendicular to the scanning line (Y direction) and connecting measurement results, is referred to as “C scan”. In a case where the number of the B-scans performed for each C-scan is Ny, the tomography structural data acquired by the C-scan are three-dimensional luminance data indicating the object light backscatter intensity at Nz×Nx×Ny points.

The signal processing unit 160 transmits digitized data to the arithmetic apparatus 21. The operation performed by the signal processing unit 160 may be performed by the arithmetic apparatus 21.

### Effect of Optical Coherence Tomography

According to the optical coherence tomography, it is possible to acquire a fingerprint image of an outer layer of the skin (or the epidermis), in a contactless manner. Since a fingertip is not brought into contact with a glass plate or the like, it is hygienic. In addition, the fingerprint image according to the optical coherence tomography is not affected by deformation at the time of the contact, unlike epidermis fingerprint imaging in which the fingertip is brought into with the glass plate or the like to capture the fingerprint image. In addition, the fingerprint image of the dermis can be acquired by the optical coherence tomography. That is, the fingerprint image can be acquired without being affected by a condition of the epidermis, so that it is possible to acquire the fingerprint image even in a case where it is hard to read the epidermis fingerprint. In addition, in a case where the epidermis fingerprint is modified, it is suitable to discover/detect the modification.

### 2-3: Information Processing Operation Performed By Information Processing Apparatus 2

With reference to FIG. 4, a flow of an information processing operation performed by the information processing apparatus 2 in the second example embodiment will be described. FIG. 4 is a flowchart illustrating the flow of the information processing operation performed by the information processing apparatus 2 in the second example embodiment.

As illustrated in FIG. 4, the detection unit 211 detects a target-to-target distance between the imaging target O and the light irradiation unit 130 that irradiates light for scanning the imaging target O (step S20). The detection unit 211 may detect a distance d between the imaging target O and the light irradiation unit 130, as illustrated in FIG. 3. The detection unit 211 may detect the target-to-target distance, on the basis of a B-scan result by the optical coherence tomography apparatus 100. The target-to-target distance may be a distance in the Z-direction. The detection unit 211 may perform the B-scan on the imaging target O located at a desired position on an XY plane, and may detect the target-to-target distance by using an analysis of two-dimensional cross-sectional image acquired by the B-scan. In a case where the target-to-target distance is detected on the basis of the B-scan result by the optical coherence tomography apparatus 100, the detection unit 211 is allowed to detect the target-to-target distance without adding a new device to the information processing apparatus 2. The detection unit 211 is also allowed to detect the target-to-target distance while continuously monitoring the imaging target O.

The moving unit 213 moves the light irradiation unit 130 to a position suitable for the scanning of the imaging target O, on the basis of the target-to-target distance (step S21). The light irradiation unit 130 is placed on a vertical movement stage, for example, and may be configured to be vertically movable. The vertical direction may be the Z direction. The moving unit 213 may vertically move the light irradiation unit 130 such that the distance between the imaging target O and the light irradiation unit 130 is an optimum distance.

The control unit 212 starts the irradiation with the light by the light irradiation unit 130 when the target-to-target distance is a desired distance (step S22). The light irradiation unit 130 may apply a light beam toward the Z direction.

### 2-4: Technical Effect of Information Processing Apparatus 2

Since the information processing apparatus 2 in the second example embodiment moves the light irradiation unit 130 to a position suitable for the scanning of the imaging target O, even when the imaging target O itself is hardly moved to an appropriate position, the light irradiation unit 130 is capable of starting the irradiation with the light while the imaging target O and the light irradiation unit 130 are in an appropriate positional relation, and it is possible to perform high-accuracy optical coherence tomography.

### 3: Third Example Embodiment

An information processing apparatus, an information processing method, and a recording medium according to a third example embodiment will be described. The following describes the information processing apparatus, the information processing method, and the recording medium according to the third example embodiment, by using an information processing apparatus 3 to which the information processing apparatus, the information processing method, and the recording medium according to the third example embodiment are applied.

### 3-1: Configuration of Information Processing Apparatus 3

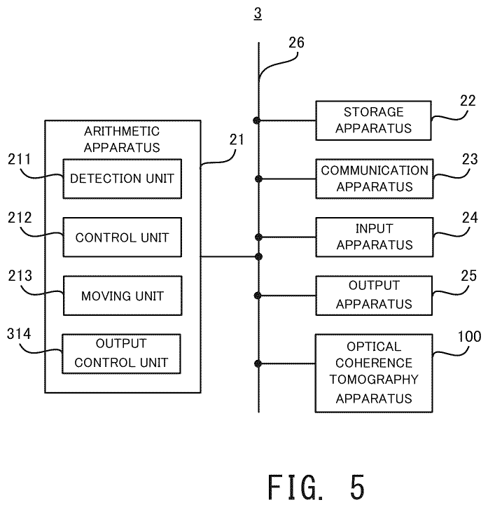

With reference to FIG. 5, a configuration of the information processing apparatus 3 in the third example embodiment will be described. FIG. 5 is a block diagram illustrating the configuration of the information processing apparatus 3 in the third example embodiment.

As illustrated in FIG. 5, the information processing apparatus 3 in the third example embodiment includes the arithmetic apparatus 21 and the storage apparatus 22, as in the information processing apparatus 2 in the second example embodiment. Furthermore, the information processing apparatus 3 may include the optical coherence tomography apparatus 100, the communication apparatus 23, the input apparatus 24, and the output apparatus 25, as in the information processing apparatus 2 in the second example embodiment. The information processing apparatus 3, however, may not include at least one of the optical coherence tomography apparatus 100, the communication apparatus 23, may not include the input apparatus 24, and the output apparatus 25. The information processing apparatus 3 in the third example embodiment is different from the information processing apparatus 2 in the second example embodiment, in that the arithmetic apparatus 21 includes an output control unit 314. Other features of the information processing apparatus 3 may be the same as those of the information processing apparatus 2 in the second example embodiment.

### 3-2: Information Processing Operation Performed By Information Processing Apparatus 3

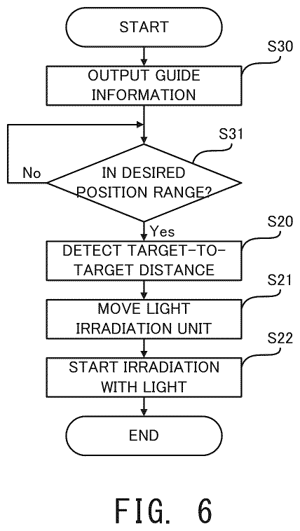

With reference to FIG. 6, a flow of the information processing operation performed by the information processing apparatus 3 in the third example embodiment will be described. FIG. 6 is a flowchart illustrating a flow of an information processing operation performed by the information processing apparatus 3 in the third example embodiment.

As illustrated in FIG. 6, the output control unit 314 outputs guide information for guiding the imaging target O to a desired position (step S30). The guide information may include at least one of visual information, auditory information, and tactile information. The output control unit 314 controls the output apparatus 25 to output the guidance information. In the third example embodiment, the guide information includes at least the visual information, and the visual information may be an image/picture visibly shown at a desired position. The output control unit 314 may display an aerial button B, as the guide information for guiding the imaging target O to a desired position.

### 3-3: Aerial Button B

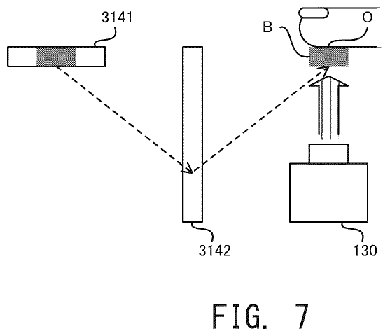

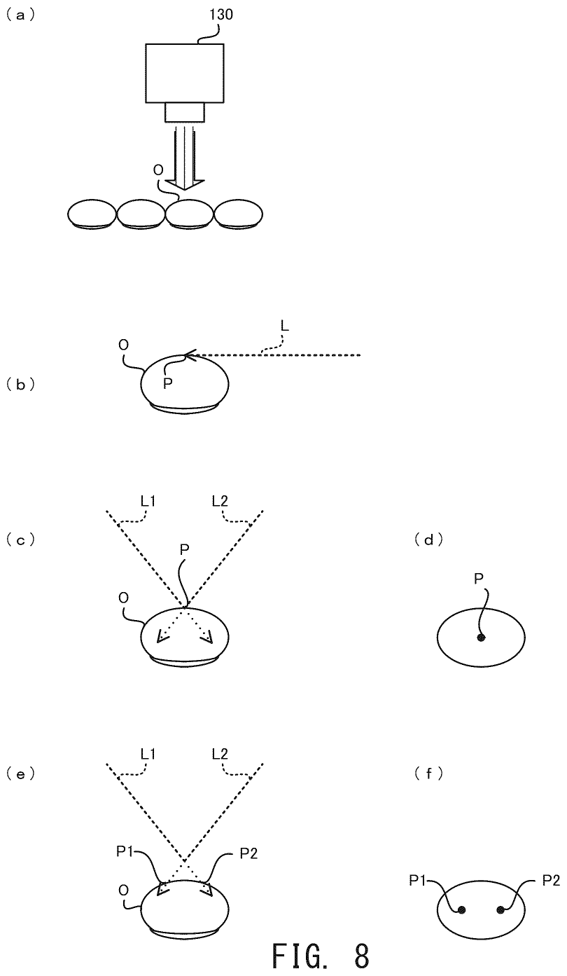

FIG. 7 is a conceptual diagram illustrating the aerial button B. As illustrated in FIG. 7, in the information processing apparatus 3 in the third example embodiment, an aerial button display 3141 for displaying the aerial button and a retroreflective plate 3142 may be provided near the information processing apparatus 3 in order to realize the aerial button B. The aerial button display 3141 may be a liquid crystal display, an OLED (Organic Light Emitting Diode) display, or the like. The retroreflective plate 3142 may be a member that allows retroreflection.

The aerial button B may be an image in which an image displayed by the aerial button display 3141 is retroreflected to the retroreflective plate 3142 and is formed in the air. The information processing apparatus 3 employs the aerial button display 3141 and the retroreflective plate 3142, thereby to form an image in the air.

The aerial button display 3141 and the retroreflective plate 3142 may be provided in accordance with a positional relation with the light irradiation unit 130. By properly providing the aerial button display 3141 and the retroreflective plate 3142, it is possible to form an image at a desired position. The output control unit 314 may display the aerial button B in the air, at an optimum position for imaging the imaging target O. The output control unit 314 may form the aerial button B over the light irradiation unit 130. The aerial button B may be used to make a finger follow it and guide the finger to a desired position. The output control unit 314 may form a button-shaped image as the aerial button B.

The detection unit 211 detects the target-to-target distance between the imaging target O and the light irradiation unit 130. The output control unit 314 may change at least one of a color and a shape of the aerial button B in accordance with a detection result by the detection unit 211. At this time, the output control unit 314 may change the color, as the imaging target approaches a target position. For example, the output control unit 314 may color the aerial button B blue in a state where there is no imaging target O, may color the aerial button B red in a state where the imaging target O is not at a position suitable for the imaging, and may color the aerial button B green in a state where the imaging target O is at a position suitable for the imaging. Furthermore, the output control unit 314 may change the color of the aerial button B, not gradually, but in gradation corresponding to the target-to-target distance.

The aerial button B may be a stereoscopic 3D image. The output control unit 314 may deform the aerial button B, in accordance with the detected result, i.e. a position of the imaging target O. The output control unit 314 may deform the shape of the aerial button B such that the button is pressed and recessed, in accordance with a vertical movement of the imaging target O detected by the detection unit 211. The output control unit 314 may deform the aerial button B such that a place over which the imaging target O is held is recessed. The output control unit 314 may deform the aerial button B and may guide the imaging target O to a more appropriate position in a case where the position of the imaging target O fits a position of the aerial button B in the in-plane direction (XY direction). The output control unit 314 may guide the imaging target O to an accurate position, by causing the measurement target person to push the button. The output control unit 314 may also discontinuously or continuously change the shape of the aerial button B.

The output control unit 314 may further output at least one of the auditory information and the tactile information, as the guide information for guiding the imaging target O to a desired position. The output control unit 314 may output, as the auditory information, for example, a sound whose pitch varies depending on the target-to-target distance, a sound whose volume varies depending on the target-to-target distance, or the like. The output control unit 314 may output a predetermined sound effect in a case where the imaging target O reaches the exact position. The output control unit 314 may output, as the auditory information, for example, a sound or voice that specifically give instruction of left, right, up, down, and the like. The output control unit 314 may output, as the tactile information, for example, a wind whose direction varies depending on the target-to-target distance, a wind whose strength varies depending on the target-to-target distance, or the like. The output control unit 314 may output, as the tactile information, for example, an ultrasonic wave whose wavelength varies depending on the target-to-target distance, or the like.

As illustrated in FIG. 6, the detection unit 211 determines whether or not the imaging target O is in a desired position range (step S31). When the imaging target O is not in the desired position range (the step S31: No), the operation proceeds to the step S31.

When the imaging target O is in the desired position range (the step S31: Yes), the detection unit 211 detects the target-to-target distance between the imaging target O and the light irradiation unit 130 that irradiates light for scanning the imaging target O (step S20). The moving unit 213 moves the light irradiation unit 130 to a position suitable for the scanning of the imaging target O, on the basis of the target-to-target distance (step S21). The control unit 212 starts the irradiation with the light by the light irradiation unit 130 when the target-to-target distance is a desired distance (step S22).

In the optical coherence tomography, it is preferable that the positional relation between the imaging target O and the light irradiation unit 130 is appropriate. That is, in the optical coherence tomography, it is preferable that the imaging target O and the light irradiation unit 130 properly allow relative movement/displacement. The information processing apparatus 3 may allow the guidance information to guide and move the imaging target O, or may move the optical illumination unit 130 on the basis of the target-to-target distance. The information processing apparatus 3 may perform at least one of an output operation of the output control unit 314 and a moving operation by the moving unit 213, thereby to perform an adjustment such that the imaging target O and the light irradiation unit 130 are in an appropriate positional relation.

By the light irradiation unit 130 applying the light perpendicularly to the imaging target O, it is possible to accurately perform the optical coherence tomography. That is, it is preferable that the positions of the imaging target O and the light irradiation unit 130 are the same in a plane in the direction perpendicular to an irradiation direction of the light by the light irradiation unit 130. The output operation by the output control unit 314 makes it possible to lead to a positional relation in which the light is applied perpendicularly to the imaging target O.

In addition, in a human's sense of vision (or perceived distance, sensory distance), accuracy in the distance direction (Z direction) is lower than that in the in-plane direction (XY direction) in many cases. Therefore, especially with respect to the movement in the XY direction, the information processing apparatus 3 may allow the guidance information to guide and move the imaging target O. Furthermore, especially with respect to the movement in the Z-direction, the information processing apparatus 3 may move the light irradiation unit 130 on the basis of the target-to-target distance.

### 3-4: Technical Effect of Information Processing Apparatus 3

There are many situations in which on feels reluctant to directly touch an object that are touched by a large number of unspecified people. There is also such a social problem that fingerprints remaining on a contact part by a contact operation are stolen. For the purpose of considering such sanitary aspects and reducing security risks, a non-contact operation is required in various input devices. In the non-contact operation, however, it is harder to control to perform the operation in an appropriate positional relation, than in the contact operation.

In contrast, since the information processing apparatus 3 in the third example embodiment displays a visibly shown image in a space in which there is no entity, it is possible to guide the imaging target to a desired position in a completely non-contact manner. Furthermore, since the information processing apparatus 3 changes at least one of the color and the shape of the image in accordance with the target-to-target distance, it is possible to assist the imaging target O to easily move to an appropriate position.

Since the information processing apparatus 3 in the third example embodiment guides the imaging target O in the in-plane direction and in the distance direction, and further mechanically adjusts the distance direction, it is possible to obtain an appropriate positional relation with high accuracy.

### 4: Fourth Example Embodiment

An information processing apparatus, an information processing method, and a recording medium according to a fourth example embodiment will be described. The following describes the information processing apparatus, the information processing method, and the recording medium according to the fourth example embodiment, by using an information processing apparatus 4 to which the information processing apparatus, the information processing method, and the recording medium according to the fourth example embodiment are applied.

The information processing apparatus 4 in the fourth example embodiment is different from the information processing apparatus 3 in the third example embodiment, in the output operation by the output control unit 314. Other features of the information processing apparatus 4 may be the same as those of the information processing apparatus 3.

### 4-1: In Case of Optical Coherence Tomography of Image Target O From Above

FIG. 8 is a conceptual diagram illustrating a case where the light irradiation unit 130 performs the optical coherence tomography on the imaging target O from above. As illustrated in FIG. 8(a), the light irradiation unit 130 may be configured to apply the light to the imaging target O located below the light irradiation unit 130, from above.

As illustrated in FIG. 8(b), the output control unit 314 may output one guiding light beam to a desired position P from the side, as the guide information guide information for guiding the imaging target O to a desired position. The measurement target person may move the finger such that the guiding light beam is applied to the center of a ball of the finger.

As illustrated in FIG. 8(c) and FIG. 8(e), the output control unit 314 may output two guiding light beams to the desired position P, as the guide information guide information for guiding the imaging target O to a desired position. The output control unit 314 may identify the desired one position P by using the two guiding light beams. FIG. 8(d) and FIG. 8(f) illustrate how it looks when the imaging target O is observed from above. As illustrated in FIG. 8(c) and FIG. 8(e), in a case where the imaging target O is positioned at a desired level/height, there is one point formed by the optical beams, with a reference numeral of P. In contrast, as illustrated in FIG. 8(e) and FIG. 8(f), in a case where the imaging target O is deviated from the desired level/height, there are two points formed by the optical beams, with reference numerals of P1 and P2. The measurement target person may move the finger such that a single guiding light beam is applied to the center of the ball of the finger. The output control unit 314 may guide the imaging target O to a position where there is exactly one point.

### 4-2: In Case of Optical Coherence Tomography of Image Target O From Below

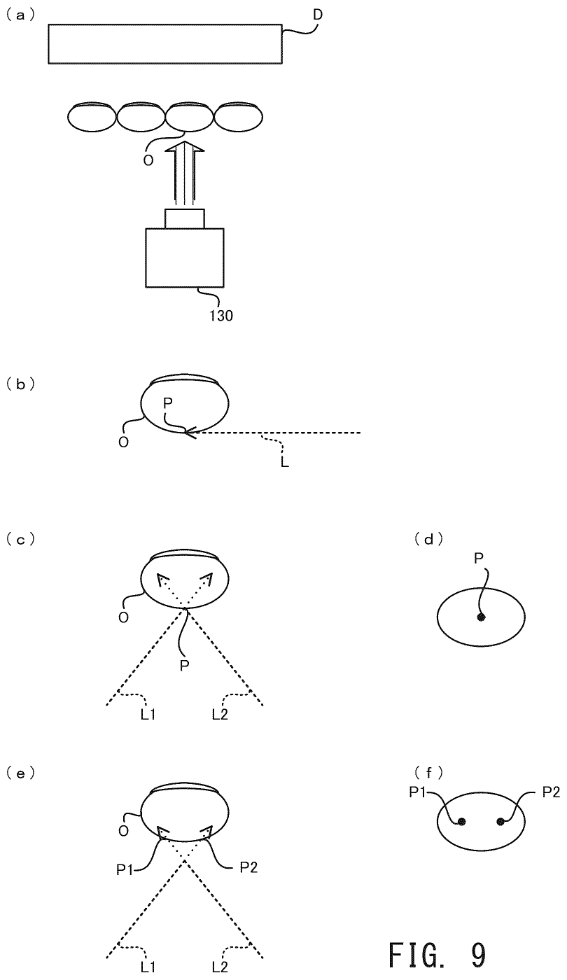

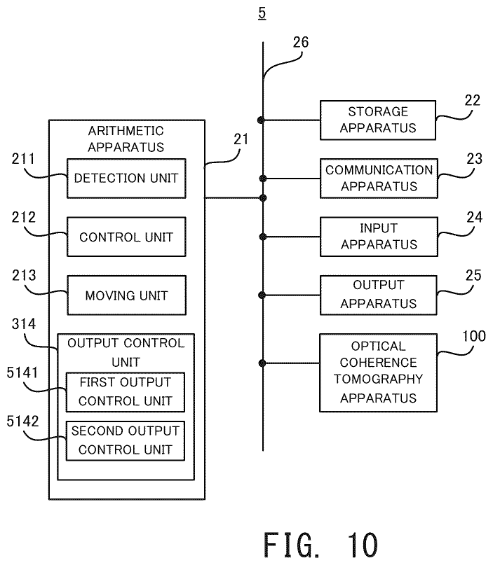

FIG. 9 is a conceptual diagram illustrating a case where the light irradiation unit 130 performs the optical coherence tomography on the imaging target O from below. As illustrated in FIG. 9 (a), the light irradiation unit 130 may be configured to apply the light beam to the imaging target O located above the light irradiation unit 130, from below.

As illustrated in FIG. 9(b), the output control unit 314 may output one guiding light beam to the desired position P from the side, as the guide information guide information for guiding the imaging target O to a desired position. The measurement target person may move the finger such that the guiding light beam is applied to the center of the ball of the finger.

As illustrated in FIG. 9(c) and FIG. 9(e), the output control unit 314 may output two guiding light beams to the desired position P, as the guide information guide information for guiding the imaging target O to a desired position. The output control unit 314 may identify the desired one position P by using the two guiding light beams. FIG. 9(d) and FIG. 9(f) illustrate how it looks when the imaging target O is observed from below. As illustrated in FIG. 9(c) and FIG. 9(e), in a case where the imaging target O is positioned at a desired level/height, there is one point formed by the optical beams, with a reference numeral of P. In contrast, as illustrated in FIG. 9(e) and FIG. 9(f), in a case where the imaging target O is deviated from the desired level/height, there are two points formed by the optical beams, with reference numerals of P1 and P2. The measurement target person may move the finger such that a single guiding light beam is applied to the center of the ball of the finger. The output control unit 314 may guide the imaging target O to a position where there is exactly one point.

Furthermore, as illustrated in FIG. 9(a), the information processing apparatus 4 may image the imaging target O from below by using a not-illustrated camera, and may display an imaging result by the camera on a display D disposed with its display surface facing upward. The imaging result may be an optical coherence tomography image. The display D may display the ball of the finger facing downward and the beam. The display D may display the target-to-target distance between the imaging target O and the light irradiation unit 130. The camera may be a stereo camera, and an image displayed on the display D may be a stereo image.

The output control unit 314 may further output at least one of the auditory information and the tactile information, as the guide information guide information for guiding the imaging target O to a desired position. The output control unit 314 may output, as the auditory information, for example, a sound whose pitch varies depending on the target-to-target distance, a sound whose volume varies depending on the target-to-target distance, or the like. The output control unit 314 may output, as the auditory information, for example, a sound or voice that specifically give instruction of left, right, up, down, and the like. The output control unit 314 may output a predetermined sound effect in a case where the imaging target O reaches the exact position. The output control unit 314 may output, as the tactile information, for example, a wind whose direction varies depending on the target-to-target distance, a wind whose strength varies depending on the target-to-target distance, or the like. The output control unit 314 may output, as the tactile information, for example, an ultrasonic wave whose wavelength varies depending on the target-to-target distance, or the like.

In the third example embodiment and the fourth example embodiment, described is the case where the output control unit 314 outputs the visual information as the guide information guide information for guiding the imaging target O to a desired position, but the output control unit 314 may not output the visual information as the guide information, but may output at least one of the auditory information and the tactile information.

### 4-3: Technical Effect of Information Processing Apparatus 4

Since the information processing apparatus 4 in the fourth example embodiment guides the imaging target to a desired position by using at least one of the visual information, the auditory information, and the tactile information, it is possible to perform the optical coherence tomography while the imaging target O and the light irradiation unit 130 are in an appropriate positional relation.

### 5: Fifth Example Embodiment

An information processing apparatus, an information processing method, and a recording medium according to a fifth example embodiment will be described. The following describes the information processing apparatus, the information processing method, and the recording medium according to the fifth example embodiment, by using an information processing apparatus 5 to which the information processing apparatus, the information processing method, and the recording medium according to the fifth example embodiment are applied.

### 5-1: Configuration of Information Processing Apparatus 5

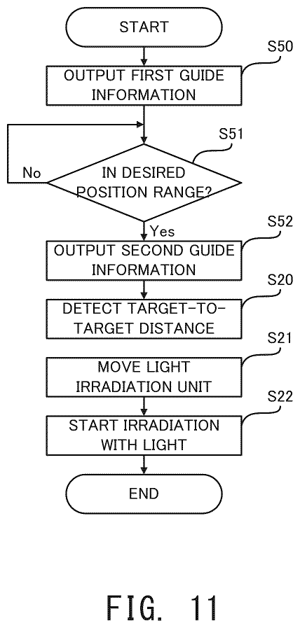

With reference to FIG. 10, a configuration of the information processing apparatus 5 in the fifth example embodiment will be described. FIG. 10 is a block diagram illustrating the configuration of the information processing apparatus 5 in the fifth example embodiment.

As illustrated in FIG. 10, the information processing apparatus 5 in the fifth example embodiment includes the arithmetic apparatus 21 and the storage apparatus 22, as in the information processing apparatus 3 in the third example embodiment and the information processing apparatus 4 in the fourth example embodiment. Furthermore, the information processing apparatus 5 may include the optical coherence tomography apparatus 100, the communication apparatus 23, the input apparatus 24, and the output apparatus 25, as in the information processing apparatus 3 in the third example embodiment and the information processing apparatus 4 in the fourth example embodiment. The information processing apparatus 5, however, may not include at least one of the optical coherence tomography apparatus 100, the communication apparatus 23, the input apparatus 24, and the output apparatus 25. The information processing apparatus 5 in the fifth example embodiment is different from the information processing apparatus 3 in the third example embodiment and the information processing apparatus 4 in the fourth example embodiment, in that the output control unit 314 provided in the arithmetic apparatus 21 includes a first output control unit 5141 and a second output control unit 5142. Other features of the information processing apparatus 5 may be the same as those of at least one of the information processing apparatus 3 in the third example embodiment and the information processing apparatus 4 in the fourth example embodiment.

### 5-2: Information Processing Operation Performed By Information Processing Apparatus 51

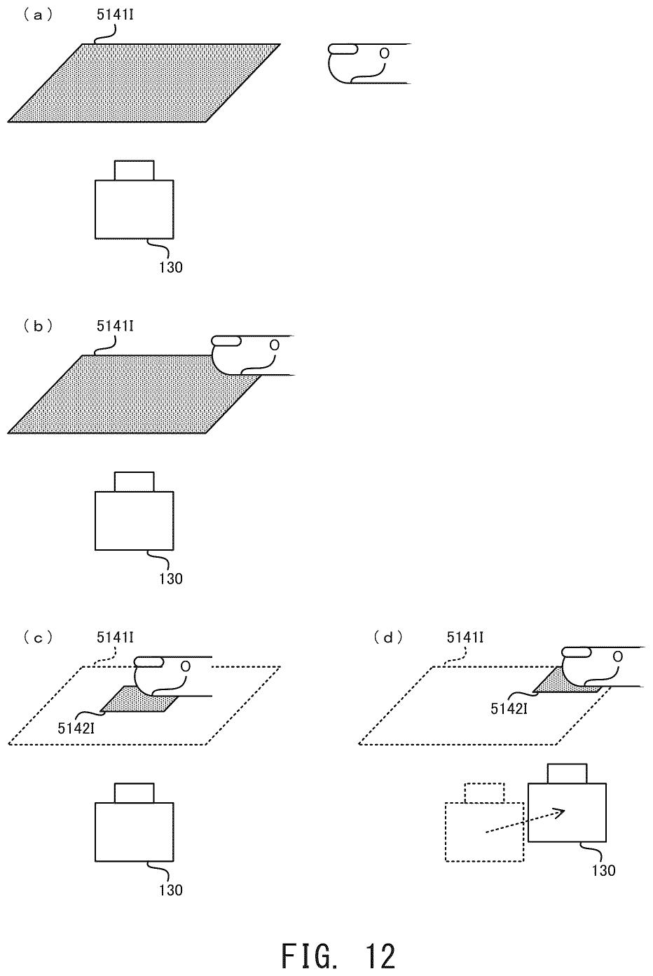

With reference to FIG. 11 and FIG. 12, a flow of the information processing operation performed by the information processing apparatus 5 in the fifth example embodiment will be described. FIG. 11 is a flowchart illustrating the flow of the information processing operation performed by an information processing apparatus 5 in the fifth example embodiment. FIG. 12 is a conceptual diagram illustrating the flow of the information processing operation performed by the information processing apparatus 5 in the fifth example embodiment.

As illustrated in FIG. 11, the first output control unit 5141 outputs first guide information for guiding the imaging target O into a desired position range (step S50). As illustrated in FIG. 12(a), the first output control unit 5141 may output a first guide image 51411 serving as the first guide information that attracts attention of the measurement target person so as to move the imaging target O to a desired position. The first guide information may be information indicating a range that can be imaged by the light irradiation unit 130. The first guide information may be information indicating a movable range of the light irradiation unit 130 in the XY plane.

The detection unit 211 determines whether or not the imaging target O is in a desired position range (step S51). When the imaging target O is not in the desired position range (the step S51: No), the operation proceeds to the step S51.

As illustrated in FIG. 12(b), when the imaging target O is in the desired position range (the step S51: Yes), the second output control unit 5142 outputs second guide information for guiding the imaging target O to a desired position (step S52). As illustrated in FIG. 12(c), the second output control unit 5142 may output a second guide image 51421 serving as the second guide information for exactly fitting the imaging target O with a desired position. The second guide information may be a shape such as a U-shape (or a concave shape), so as to guide not only a position of the finger, but also a direction of the finger. In the fifth example embodiment, the output control unit 314 may operate the first output control unit 5141 at the start of the operation, and may switch the operation of the first output control unit 5141 to the operation of the second output control unit 5142 in a case where the imaging target enters the desired position range.

The detection unit 211 detects the target-to-target distance between the imaging target O and the light irradiation unit 130 that irradiates light for scanning the imaging target O (step S20). The moving unit 213 moves the light irradiation unit 130 to a position suitable for the scanning of the imaging target O, on the basis of the target-to-target distance (step S21). The control unit 212 starts the irradiation with the light by the light irradiation unit 130 when the target-to-target distance is a desired distance (step S22).

As illustrated in FIG. 12(d), when the imaging target O is in the desired position range (the step S51: Yes), in a step S52, the second output control unit 5142 may output the second guide information to a position corresponding to the position of the imaging target O, and the moving unit 213 may move the light irradiator 130 to the position corresponding to the position of the imaging target O. That is, the second guide information may be information indicating a range that can be imaged by the light irradiation unit 130 after moving in the XY direction.

### 5-3: Technical Effect of Information Processing Apparatus 5

The information processing apparatus 5 in the fifth example embodiment allows the measurement target person to perform rough motions and fine motions, by outputting the guidance information that i divided into stages.

### 6: Sixth Example Embodiment

An information processing apparatus, an information processing method, and a recording medium according to a sixth example embodiment will be described. The following describes the information processing apparatus, the information processing method, and the recording medium according to the sixth example embodiment, by using an information processing apparatus 6 to which the information processing apparatus, the information processing method, and the recording medium according to the sixth example embodiment are applied.

The information processing apparatus 6 in the sixth example embodiment is different in an initial position of the light irradiation unit 130, from the information processing apparatus 2 in the second example embodiment to the information processing apparatus 5 in the fifth example embodiment. Other features of the information processing apparatus 6 may be the same as those of at least one of the information processing apparatus 2 to the information processing apparatus 5.

In the sixth example embodiment, the initial position of the light irradiation unit 130 is determined on the basis of statistical information on the position of the imaging target O at the start of the operation of the detection unit 211 in the past. For example, a position where the imaging target O is likely to exist, may be biased due to a posture that is easily taken by the measurement target person, or the like. The moving unit 213 may store, in the storage apparatus 22, positions where a plurality of measurement target persons firstly hold the fingers over the light irradiation unit 130, and the position of the light irradiation unit 130 at the start of the irradiation with the light by the light irradiation unit 130. The moving unit 213 may move in advance the light irradiation unit 130 before the start of the information processing operation, to a position where the finger is statistically likely to be firstly held over. The moving unit 213 may determine the initial position of the light irradiation unit 130 on the basis of history information on the position where the measurement target person firstly holds the finger over the light irradiation unit 130.

Furthermore, the moving unit 213 may associate the initial position of the light irradiation unit 130 with a time zone and day of the week. The moving unit 213 may determine the initial position in accordance with the day of the week, the time zone, or the like. Depending on the day of the week and the time zone, the position where the imaging target is likely to exist, is biased in many cases. That is, a method of setting the initial position may be changed arbitrarily in accordance with an environment used.

The information processing apparatus 6 in the sixth example embodiment determines the initial position of the light irradiation unit 130 on the basis of the statistical information on the position of the imaging target O at the start of the detection operation in the past, thereby to reduce an amount of movement of the light irradiation unit 130 and to reduce an operation load.

### 7: Supplementary Notes

With respect to the example embodiment described above, the following Supplementary Notes are further disclosed.

### Supplementary Note 1

An information processing apparatus including:
a detection unit that detects a target-to-target distance between an imaging target and a light irradiation unit that irradiates light for scanning the imaging target; and
a scan control unit that starts irradiation with the light by the light irradiation unit in a case where the target-to-target distance is a desired distance.

### Supplementary Note 2

The information processing apparatus according to Supplementary Note 1, further including a moving unit that moves the light irradiation unit to a position suitable for scanning of the imaging target on the basis of the target-to-target distance.

### Supplementary Note 3

The information processing apparatus according to Supplementary Note 1 or 2, further including an output unit that outputs guide information for guiding the imaging target to a desired position, wherein the guide information includes at least one of visual information, auditory information, and tactile information.

### Supplementary Note 4

The information processing apparatus according to Supplementary Note 3, wherein the visual information is an image visibly shown at the desired position.

### Supplementary Note 5

The information processing apparatus according to Supplementary Note 4, wherein the output unit changes at least one of a color and a shape of the image in accordance with the target-to-target distance.

### Supplementary Note 6

The information processing apparatus according to any one of Supplementary Notes 3 to 5, wherein the output unit
includes:
a first output unit that outputs first guide information for guiding the imaging target into
a desired position range; and
a second output unit that outputs second guide information for guiding the imaging target to the desired position,
operates the first output unit at a start of an operation of the output unit, and
switches an operation of the first output unit to an operation of the second output unit in a case where the imaging target enters the desired position range.

### Supplementary Note 7

The information processing apparatus according to any one of Supplementary Notes 1 to 6, wherein an initial position of the light irradiation unit is determined on the basis of statistical information on a position of the imaging target at a start of an operation of the detection unit in a past.

### Supplementary Note 8

An information processing method including:
detecting a target-to-target distance between an imaging target and a light irradiation unit that irradiates light for scanning the imaging target; and
starting irradiation with the light by the light irradiation unit in a case where the target-to-target distance is a desired distance.

### Supplementary Note 9

A recording medium on which a computer program that allows a computer to execute an information processing method is recorded, the information processing method including:
detecting a target-to-target distance between an imaging target and a light irradiation unit that irradiates light for scanning the imaging target; and
starting irradiation with the light by the light irradiation unit in a case where the target-to-target distance is a desired distance.

At least a part of the constituent components of each of the example embodiments described above can be combined with at least another part of the constituent components of each of the example embodiments described above, as appropriate. A part of the constituent components of each of the example embodiments described above may not be used. Furthermore, to the extent permitted by law, all the references (e.g., publications) cited in this disclosure are incorporated by reference as a part of the description of this disclosure.

This disclosure is not limited to the examples described above and is allowed to be changed, if desired, without departing from the essence or spirit of this disclosure which can be read from the claims and the entire identification. An information processing apparatus, an information processing method, and a recording medium with such changes are also intended to be within the technical scope of this disclosure.

### DESCRIPTION OF REFERENCE CODES

1, 2, 3, 4, 5, 6 Information processing apparatus
11, 211 Detection unit
12, 212 Control unit
213 Moving unit
314 Output control unit
5141 First output control unit
5142 Second output control unit
100 Optical coherence tomography apparatus
110 Light source unit
120 Splitter/combiner
130 Light irradiation unit
140 Mirror unit
150 Light receiving unit
160 Signal processing unit
O Imaging target
B Aerial button
3141 Aerial button display
3142 Retroreflective plate
P Desired position

What is claimed is:

1. An information processing apparatus comprising:
at least one memory that is configured to store instructions; and
at least one first processor that is configured to execute the instructions to:
detect a target-to-target distance between an imaging target and a light irradiation unit that irradiates light for scanning the imaging target; and
start irradiation with the light by the light irradiation unit in a case where the target-to-target distance is a desired distance.

2. The information processing apparatus according to claim 1, wherein the at least one first processor is configured to execute the instructions to move the light irradiation unit to a position suitable for scanning of the imaging target on the basis of the target-to-target distance.

3. The information processing apparatus according to claim 1, wherein the at least one first processor is configured to execute the instructions to output guide information for guiding the imaging target to a desired position, and
the guide information includes at least one of visual information, auditory information, and tactile information.

4. The information processing apparatus according to claim 3, wherein the visual information is an image visibly shown at the desired position.

5. The information processing apparatus according to claim 4, wherein the at least one first processor is configured to execute the instructions to change at least one of a color and a shape of the image in accordance with the target-to-target distance.

6. The information processing apparatus according to claim 3, wherein the at least one first processor is configured to execute the instructions to
perform:
a first output operation of outputting first guide information for guiding the imaging target into a desired position range; and
a second output operation of outputting second guide information for guiding the imaging target to the desired position,
perform the first output operation at a start of an operation, and
switches the first output operation to the second output operation in a case where the imaging target enters the desired position range.

7. The information processing apparatus according to claim 1, wherein an initial position of the light irradiation unit is determined on the basis of statistical information on a position of the imaging target at a start of an operation of detecting the target-to-target distance in a past.

8. An information processing method comprising:
detecting a target-to-target distance between an imaging target and a light irradiation unit that irradiates light for scanning the imaging target; and
starting irradiation with the light by the light irradiation unit in a case where the target-to-target distance is a desired distance.

9. A non-transitory recording medium on which a computer program that allows a computer to execute an information processing method is recorded, the information processing method including:
detecting a target-to-target distance between an imaging target and a light irradiation unit that irradiates light for scanning the imaging target; and
starting irradiation with the light by the light irradiation unit in a case where the target-to-target distance is a desired distance.
